(19) United States
(12) Patent Application Publication
(10) Pub. No.: US 2021/0100673 A1
(43) Pub. Date: Apr. 8, 2021

(54) ORTHESIS, SYSTEM AND METHOD FOR ADAPTING AN ORTHESIS

(71) Applicant: Ottobock SE & Co. KGaA, Duderstadt (DE)

(72) Inventor: Gordon SIEWERT, Göttingen (DE)

(73) Assignee: Ottobock SE & Co. KGaA, Duderstadt (DE)

(21) Appl. No.: 15/733,146

(22) PCT Filed: Nov. 30, 2018
(86) PCT No.: PCT/EP2018/083134

§ 371 Date: May 28, 2020

(30) Foreign Application Priority Data
Dec. 1, 2017 (DE) 10 2017 128 618.7

(51) Int. Cl. A61F 5/01

(52) U.S. Cl. CPC A61F 5/0125; A61F 5/0127; A61F 2005/0137; A61F 2005/0169; A61F 2005/0155; A61F 2005/0158; A61F 2005/0167; A61F 2005/0144

(57) ABSTRACT

An orthesis, which has an upper part and a lower part, which are coupled to each other with a first joint device in such a way that the upper part and the lower part can pivot about a pivot axis, wherein devices for receiving a body part or a limb or for fastening the orthesis to a body part or a limb are arranged on the upper part and/or the lower part, and wherein an upper support element is fastened to the upper part on one side and a lower support element is fastened to the lower part on one side, which support elements are connected to each other with a second joint device in such a way that said support elements can pivot about the pivot axis. A bearing plate having at least one fastening device for fastening the second joint device is fastened to or formed on the upper part and/or the lower part.

The invention relates to an orthosis with an upper part and a lower part, which are coupled to each other via a first joint device in such a way as to be pivotable about a pivot axis, wherein devices for receiving a body part or a limb or for securing the orthosis to a body part or a limb are arranged on the upper part and/or the lower part, a system composed of an orthosis and of a functional element, and a method for adapting an orthosis to a patient.

Orthoses are fitted in place on a limb and generally have rails or shells with devices for securing the respective rails or shells to the limb. The rails or shells are connected to each other via a joint device, such that the orthosis can be arranged on the limb in a manner engaging over the joints. By means of an orthosis, movements can be performed, pivot angles about a joint axis can be limited, pivoting movements can be prevented, or an orientation of limbs relative to each other can be assisted or secured. In addition, orthoses can be provided with damper elements in order to damp pivoting movements about the joint axis. The damper devices can be provided with a controller, such that modified damping in the flexion direction or extension direction can be made available, depending on loads, pivot angles or sensor data.

There is the possibility of assigning an energy store to the upper part and the lower part, such that, by pretensioning of a spring or of an elastomeric element or of a hydraulic or pneumatic energy store, a resistance to a pivoting movement for example is made available in a first direction and is increased, wherein the opposite movement is assisted by relaxation of the energy store. End stops for setting the respective pivot angles can be formed adjustably in the joint devices, and a damper for avoiding an undamped impact in an end stop can likewise be provided.

In addition, there are orthoses with active drives in which a displacement of the upper part relative to the lower part about the pivot axis is actively effected, for example, via an electric motor. The orthoses can engage over only one joint, for example the knee joint or the ankle joint, although it is likewise possible that the orthosis engages over several joints, for example the knee joint and the ankle joint, or also the hip joint, the knee joint and the ankle joint. In an orthosis that engages over several natural joints, the proximal component is the upper part, and the distal component is the lower part. In a knee-ankle orthosis, a lower-leg rail is therefore the lower part, as regards the part engaging over the knee, since the lower-leg rail is arranged distally with respect to a thigh rail. With respect to a foot rail or foot shell, the lower-leg rail is then an upper part, while the foot rail or foot shell, which is connected to the lower-leg rail via an orthosis ankle joint, forms the lower part.

In highly loaded orthoses, for example in knee-ankle-foot orthoses, which are known by the abbreviation KAFO, joints are provided bilaterally in the knee region in order to configure an orthosis. A joint device is arranged both on the medial side and also on the lateral side of the knee joint, said joint devices each having a thigh rail and a lower-leg rail, which are connected to each other. Problems posed by a bilateral arrangement of rails on a leg include the difficulty in adapting to different leg circumferences, the interference caused by the medial joint device, which can collide with the unaffected leg, and complicated adaptation, particularly in trial set-ups.

The object of the present invention is therefore to make available an orthosis, a system composed of an orthosis and of a functional element, and a method, which do not have the disadvantages of the prior art. According to the invention, this object is achieved by an orthosis having the features of the main claim, and by a system and a method having the features of the additional independent claims. Advantageous embodiments and developments of the invention are set forth in the dependent claims, the description and the figures.

In the orthosis according to the invention with an upper part and a lower part, which are coupled to each other via a first joint device in such a way as to be pivotable about a pivot axis, wherein devices for receiving a body part or a limb or for securing the orthosis to a body part or a limb are arranged on the upper part and/or the lower part, provision is made that an upper support element is secured to the upper part on one side, in particular laterally, and a lower support element is secured to the lower part on one side, in particular laterally, which support elements are connected to each other via a second joint device in such a way as to be pivotable about the pivot axis, wherein a bearing plate having at least one fastening device for securing the second joint device is fastened to or formed on the upper part and/or the lower part. By the arrangement of the two support elements on one side, in particular laterally on the upper part and the lower part, if appropriate also on rail parts or components which are arranged or formed on the upper part and lower part, the torsional moments and the moments arising in the orthosis are better taken up in a frontal plane. The functionality of the orthosis is not limited here, since the second joint device, which connects the upper and lower support element pivotably to each other, permits a pivoting movement about a common pivot axis of the two joint devices. The pivot axes of the joint devices are coaxial, wherein the pivot axis preferably runs coaxially with respect to the natural joint axis or a compromise joint axis, so as to permit a movement of the natural limb that is impeded as little as possible. The second joint device in particular can have a virtual joint axis, and it is therefore only necessary that the upper and lower support element are able to pivot about a common pivot axis with the upper part and lower part. A bearing plate is fastened to or formed on the upper part and/or the lower part and has at least one fastening device to which the second joint device can be secured. By way of the bearing plate and the fastening device, it is possible to fasten different joint devices to the upper part and/or the lower part, without having to make adjustments to the second joint device, in particular the upper support element and the lower support element. In this way, the modular set-up of the orthosis can be facilitated, and, in particular, a selection can be made from a multiplicity of different second joint devices in order, for example, to carry out an assembly for test purposes. Particularly if several fastening devices are arranged or formed at different locations on the bearing plate, the assembly of different second joint devices is made easier. It is no longer necessary to secure the upper and lower support element directly on the upper part or the lower part, and instead the securing can be done substantially independently of the configuration of the upper part and/or the lower part. The fastening devices can be arranged displaceably on the bearing plate. The adaptability to different second joint devices can be increased by displaceable fastening devices, for example screw inserts, bolts, lockable slide guides or the like. The bearing plate as such does not have to be flat; it can likewise have a curvature. Bearing plates can be arranged both on the upper part and also on the lower part; alternatively, a bearing plate is arranged either on the upper part or on the lower part and serves to secure the second joint device. In the case where there is just one bearing plate, the respective other support element, which is not secured to the bearing plate, is secured to the upper part or the lower part respectively. The bearing plate is an adapter which is arranged on the first joint device and via which the fastening of the second joint device can take place or is made easier.

On account of the arrangement of the additional support elements on one side, in particular laterally with respect to the upper part and the lower part, and on account of the resulting arrangement of the upper part and lower part on one side, in particular laterally, alongside the natural limb, the opposite region of the limb, in particular the medial region, is free, in particular when the orthosis is used as an orthosis of a lower extremity, such that a slim configuration is achieved, and, in the case of a lateral arrangement, no collisions of orthosis components with the contralateral side or the torso can occur. If both limbs are fitted with such a laterally arranged orthosis, in particular the lower limbs, more space for swing-through of the lower limb is present on the medial side, which increases the wearing comfort.

The orthosis can have devices for receiving a body part or a limb or fastening devices for fastening the orthosis to a body part or a limb. Orthoses are often secured to the orthosis user by a strap system or a combination of a shell receptacle with a strap system. The fact that the double joint device and therefore the rails or the shells extending from the joint device along the limb are arranged on one side, in particular only laterally, facilitates the adaptation of the orthosis to different limb circumferences, with the result that such an orthosis can be used very effectively as a test orthosis, on which the final set-up of the individual components can be arranged or changed around for optimal patient care.

In a development of the invention, provision is made that the first joint device and the second joint device are arranged laterally and spaced apart from each other. By virtue of the joint devices being spaced apart from each other, a circumferential frame structure is obtained via which torsional moments can be better taken up along the longitudinal extent of the orthosis. Similarly, in the case of a relatively wide configuration, the moments in the frontal plane can be better taken up by virtue of a spacing between the two joint devices, resulting in a more stable configuration of the orthosis, even when the joint devices are arranged only laterally on a limb.

The support elements can be secured to each other releasably and connected to each other via the second joint device, so as to be able to more easily exchange the individual components. Thus, different upper support elements can be releasably connectable to just one lower support element, such that different geometries, dimensions or connections can be made available in the upper support element and can be easily changed if this proves necessary for the adaptation to the particular patient. Thus, with a base orthosis and different upper and lower support elements that are able to be combined with one another, it is possible to make available a large number of individually adaptable orthoses.

Fastening devices for the releasable fastening of rails or devices for receiving a body part or a limb can be formed or arranged on the upper part and the lower part. The fastening devices are preferably what are known as rail boxes, or receptacles for rails, which are secured in the upper part with form-fit engagement, for example by screws or by similar releasable form-fit elements. It is thus possible for different rails or devices for receiving a body part or a limb, for example shells, foot supports or the like, to be variably fastened to the upper part or lower part in order to achieve simple adaptation to the requirements of the users.

Receptacles for a damper, a drive, a control device and/or a sensor can be arranged or formed on the upper support element and the lower support element, so as to be able to expand the functionality of the orthosis via the support elements. The upper and lower support element serves as a carrier for further components, for example damper devices or drives, in order to influence the pivoting movement of the orthosis. Purely passive add-on components such as damper devices or springs can be mounted on the orthosis via the support elements, such that the support elements are used as module carriers. The damper device can be adjustable, in particular with an adjustability based on sensor data, for which purpose an adjusting mechanism with a controller has to be present inside the damper device, so as to permit an adjustment of the damping on the basis of sensor data. A drive, in particular a motorized drive or an actuator driven by a pump, can likewise be fastened to the orthosis via the upper and lower support element, such that a movement of the limb is assisted or indeed made possible via a motorized drive. The motorized drive can at the same time be used as a damper. The support elements have suitable receptacles for the components that are to be inserted, which can be screwed onto the support elements or can be coupled thereto in some other way so as to transmit force, in particular with form-fit engagement, in particular spring stores, extension assists, enabling and locking devices, actuators, energy stores, pumps, motors, dampers, control devices and/or sensors.

The support elements are preferably fastened releasably to the upper part and the lower part via the bearing plate, so as to ensure easy exchangeability of the support elements. On account of the lateral arrangement both of the upper support element and of the lower support element, an exchange can easily take place, even with the orthosis fitted, in order to facilitate adaptation to the particular user. The bearing plate itself can preferably be fastened exchangeably on the upper part or lower part, so as to be able to mount different bearing plates, which can have different fastening devices or which have fastening devices at different locations or in different arrangements.

The upper and/or lower support element can be designed to be adjustable in terms of angle to the pivot axis, such that tilting is possible in the frontal plane in an extended position of the orthosis. This tilting does not adversely affect the coaxial nature of the pivot axes and may be necessary in order to permit optimized adaptation to the particular conditions of use.

The orthosis is preferably designed as a unilateral orthosis of a lower extremity, in particular as a cross-knee orthosis, a knee-ankle-foot orthosis, a hip-knee-ankle-foot orthosis or an ankle-foot orthosis, since orthoses of the lower extremity have to take up and transmit comparatively high forces and moments, which can be taken up effectively via the second joint device. For this purpose, it is advantageous if the support elements are secured to the upper part and the lower part to transmit force and moment. In particular, the support elements are screwed onto the upper part and the lower part, if appropriate inserted into preformed projections or recesses, in order to ensure a fixed relationship and therefore a transmission of force and moment. The support elements can be made from metal or a fiber-reinforced plastic, in order to ensure sufficient strength and stability for transmitting the forces and moments acting in the orthosis.

In a development of the invention, at least one of the support elements is designed as a functional element, or such a functional element is fastened to at least one support element. The functional element can be designed as a drive, damper, brake, locking device, transducer and/or energy store. The drive can be an electromotive drive or hydraulic drive, wherein the hydraulic drive can be coupled to a pump and/or an energy store, for example a mechanical or pneumatic spring. A hydraulic volume can be subjected to pressure via the drive or the energy store in order to move a piston, for example a linear piston or a pivot piston, so as to move one orthosis component relative to the other orthosis component. It is thereby possible to pivot an upper part of the joint device relative to the lower part in order to bring about or at least assist a flexion and/or extension. It is additionally possible to configure the functional element as a damper device, in particular as a hydraulic damper in which the pivoting movement in the extension direction and/or flexion direction can be influenced by the change of a hydraulic resistance. A brake or an adjustable limit stop can likewise be formed as functional element or can be part of the functional element. In addition, the functional element can be designed as or have a transducer, in order to detect motion data and/or load data of the upper part, the lower part or further components of the orthosis or of an orthosis system. The transducers can be configured as force sensors, angle sensors, travel sensors, moment sensors or position sensors. In addition, the transducers can receive motion data such as accelerations or speeds, in order to implement these via a control device for changing resistances or settings on a functional element, for example a damper device or a drive. Depending on values detected via the transducers, the orthosis can then be controlled in order to be able to permit settings adapted to the user or to the particular movement or movement situation. An energy store as functional element is configured in particular as a spring, such that, for example, kinetic energy can be converted to deformation energy during braking of a movement and stored and, at a later point, released again in a controlled manner. This dispenses with the conversion of kinetic energy to electrical energy and with the storage of electrical energy in an accumulator. Besides an integral configuration of the support element as functional element, it is possible that such a functional element is fastened to the at least one support element in order not only to guide the limb but also to be able to analyze and/or influence the particular movement.

In the system according to the invention, composed of an orthosis as described above and of a multiplicity of different functional elements, for example different drives, different dampers, brake devices, transducers and/or springs, provision is made that these different functional elements can be secured to the same fastening devices via at least one bearing plate on the upper part and/or lower part. Besides different functional elements that perform different functions from one another, variants of functional elements with identical functions can also be mounted, tested and adapted. By way of the fastening devices on the bearing plate, the different functional elements can be easily exchanged, such that it is possible, by virtue of the modular configuration of the functional elements and the standardized arrangement and orientation of the fastening devices of the functional elements to the fastening devices on the bearing plate, to achieve easy exchangeability and therefore easy adaptability to the particular patient or to the particular purpose of use. The functional elements can be selected depending on the patient, on the purpose of use and/or on other peripheral conditions of treatment and can be adapted to a standardized orthosis. The functional elements then allow the orthosis to be tailored individually and adapted to the particular user. For this purpose, provision is made that on each functional element that is part of the system, fastening devices or fastening elements are arranged or formed which are positioned on the bearing plate for the upper part and/or lower part in a manner corresponding to the fastening devices or receiving devices provided for the purpose. For example, if a functional element configured as a passive hydraulic damper is replaced by a functional element configured as a hydraulic drive, it is possible for a passive orthosis to be made an active orthosis, by which movements can be initiated, executed or assisted. Instead of a functional element that influences the movement of the orthosis, it is likewise possible to arrange what is purely a detection unit by which motion data are detected, for example in order to be able to document progress in healing or to document the course of a physical limitation.

In a development of the system according to the invention, provision is made that all of the functional elements have fastening elements which are arranged corresponding to fastening devices for the fastening elements for securing to the upper part and/or the lower part via the bearing plate. The corresponding arrangement of the fastening elements to the receiving devices for the fastening elements permits in particular the arrangement of at least one spacer element between the actual orthosis and the functional element in order to ensure that the individual adaptability to the respective patient is ensured. For example, in the case of lower extremities, genu valgum may require a different spacing of a functional element from an upper part or lower part than in the case of genu varum, such that spacer elements can be arranged between the upper part and/or lower part or the bearing plate and the functional element, particularly if the functional element has to have a special orientation or has to be oriented specifically in relation to another component.

The functional element in the system is preferably secured releasably to the bearing plate and, if appropriate, to the upper part and the lower part, in order to facilitate adaptability to the particular purpose of use or to the particular patient. The upper support element and the lower support element can be assembled to form a module, thereby permitting a modular set-up and therefore easy exchange and easy adaptability.

In the method for adapting an orthosis, of the kind described above, to a patient, wherein the orthosis is fastened to a body part or limb via devices for receiving or for securing a body part or a limb, provision is made that, after a first joint device has been selected from a multiplicity of different first joint devices and this first joint device has been connected to devices for receiving or for securing a body part of a limb, a second joint device is selected from a multiplicity of different second joint devices, and then an upper and lower support element, which are connected to each other via the selected second joint device, are connected to the upper part and the lower part of the first joint device, such that, based on a selection of different joints or joint devices and a respective permutation of the joint devices, it is possible to provide a considerable variation of different orthosis types and to adapt them to the particular patient. The modular set-up of the individual components, in particular of the first joint devices and of the second joint devices, permits easy exchange thereof. In a development of the invention, provision is made that the joint device suitable for the patient is first of all selected from a multiplicity of first joint devices and is equipped with the rails and fastening devices. After selection of a first joint device that is considered to be the most suitable one, a multiplicity of second joint devices can be secured to the first joint device via the support elements and can be tested out or exchanged, thus allowing the orthosis to be tailored individually or adapted to the particular patient, to the purpose of use or to a variable state of a patient. For the main joint, that is to say the first joint device, loads are taken up in particular in the medial-lateral direction, whereas loads in the anterior-posterior direction are taken up and led off via the second joint device. The main joint is configured in particular as a free running joint, while the second joint device can be equipped in particular with functional elements such as drives, dampers, transducers, enabling or locking devices, extension assists, springs and/or other energy stores.

Illustrative embodiments are explained in more detail below with reference to the accompanying figures, in which:

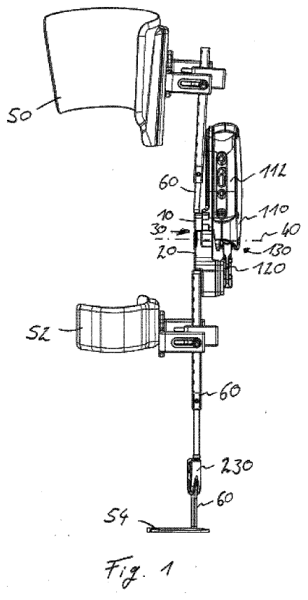

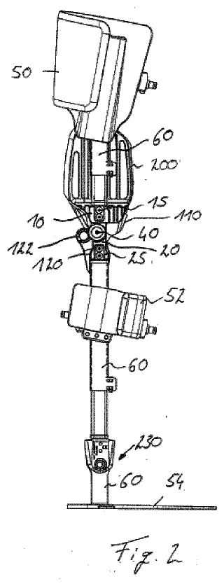

FIGS. 1-3 show a KAFO in a frontal view, a medial view and a lateral view;

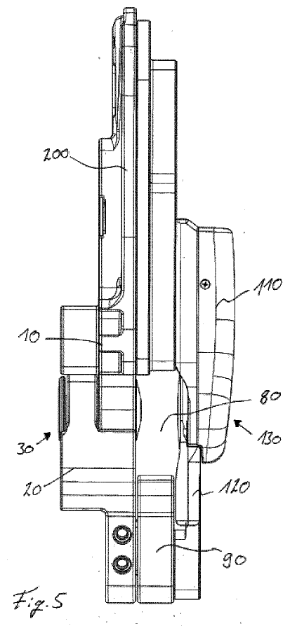

FIGS. 4-8 show a first variant of the invention in a sectional view, a frontal view, a lateral view, a rear view and a medial view;

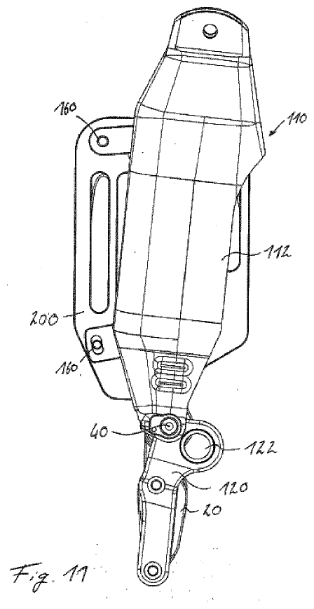

FIGS. 9-13 show a second variant in a sectional view, a frontal view, a lateral view, a rear view and a medial view;

FIGS. 14-18 show a third variant in a sectional view, a frontal view, a lateral view, a rear view and a medial view;

FIGS. 19-21 show side views of an orthosis system; and

FIGS. 19a-21a show medial side views of FIGS. 19 to 21.

FIG. 1 shows a frontal view of what is called a KAFO (knee-ankle-foot orthosis) for placing on a leg. The orthosis is secured on the leg via receiving devices 50, 52 and 54, if appropriate together with additional straps (not shown). The thigh is received in a thigh shell 50, the lower leg is received in a lower-leg shell 52, and the foot is placed on a foot plate 54. The devices 50, 52, 54 for receiving body parts are fastened to rails 60 which run along the respective limb, in the illustrative embodiment shown laterally with respect to the leg (not shown). From the thigh shell 50, a thigh rail 60 extends to an upper part 10 and is secured there with form-fit engagement, in what is called a rail box, via screws. The upper part 10 thus forms the distal endpiece of the thigh rail 60. A lower part 20 is fastened to the upper part 10 about a pivot axis 40, such that the upper part 10 together with the lower part 20 forms a joint device 30 which is arranged at the height of a natural knee joint. A lower-leg rail 60 is fastened to the lower part 20; the lower part 20 thus forms the proximal termination of the lower-leg rail 60. At the distal end of the lower-leg rail 60, an ankle joint 230 is arranged at the height of a natural ankle joint and is adjoined by a rail component 60 which connects the foot plate 54 to the orthosis ankle joint 230. The foot plate 54 is connected pivotably to the lower-leg rail 60 via the orthosis ankle joint 230.

Support elements 110, 120, arranged laterally with respect to the rails 60, are fastened to the orthosis knee joint by the joint device 30. An upper support element 110 is fastened to the upper part 10 and, if appropriate, to the thigh rail 60 so as to transmit moment and force. Correspondingly, a lower support element 120 is fastened to the lower part 20 so as to transmit force and moment, in particular by being screwed on or being secured via form-fit elements. The upper support element 110 is connected pivotably to the lower support element 120 to form a second joint device 130 pivotably, such that, spaced laterally apart from the first joint device 30 between the upper part 10 and the lower part 20, a second, parallel second joint device 130, pivotable coaxially about the pivot axis 40, is formed in the region of the orthosis knee joint. A corresponding configuration can be provided at an orthosis ankle joint or also at an orthosis hip joint.

The two support elements 110, 120 can in particular be fastened releasably to the upper part 10 and lower part 20, in order to permit easy adaptation of a base orthosis to an alternatively configured, laterally arranged second joint device 120.

A receptacle 112 for further components, for example drives, damper devices, control devices, sensors or the like, is provided in the upper support element 110.

FIG. 2 shows a medial view of the KAFO shown in FIG. 1. A rail box 15 with screws is formed on the upper part 10, in which rail box 15 the thigh rail 60 is inserted and secured with form-fit engagement. The thigh shell 50 is fastened to the thigh rail 60. Distally from the upper part 10, the lower part 20 is secured pivotably about the pivot axis 40. The lower-leg rail 60 is screwed to the lower part 20 in the rail box 25. In the medial view, the receptacle 122 can be seen in the form of a through-opening for receiving, for example, a bolt for securing a piston rod; the receiving device in the upper support element 110 cannot be seen, and the lower support element 120 can be seen only partially. Arranged on the upper part 10 is a bearing plate 200, which extends in the proximal direction past the upper part 10 and is substantially wider than the upper part 10. The upper support element 110 is secured on the bearing plate 200; the fastening of the upper support element 110 to the bearing plate 200 will later be described in detail. The lower support element 120 is releasably fastened directly on the lower part 20.

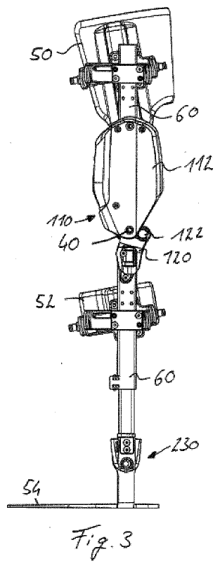

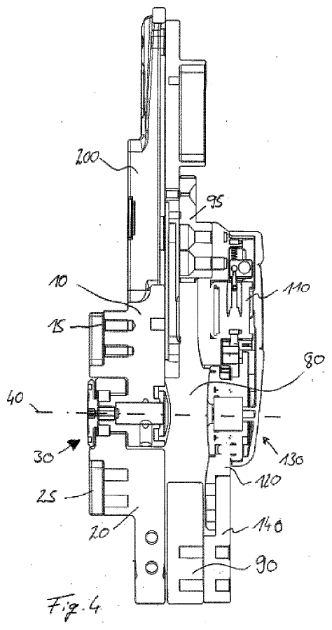

FIG. 3 shows the orthosis according to FIG. 1 in a lateral view, in which both the upper support element 100 and the lower support element 120 can be better seen. The receiving device 112 in the upper support element 110 and the receiving device 122 in the lower support element 120 can likewise be seen, also the pivotable configuration of the second joint device 130 and the coaxial configuration of the pivot axis 40 of the second joint device 130 relative to the pivot axis of the first joint device 30 of the upper part 10 and of the lower part 20. The receiving device 112, which can be a housing, completely conceals the bearing plate 200, which is located between the upper part 10 and the upper support element 110.

FIGS. 4 to 8 show individual illustrations of the configuration of the joint devices without the rails 60 and the devices 50, 52, 54 for receiving the thigh, the lower leg and the foot, in a frontal sectional view, in a front view, in a lateral view, in a rear view and in a medial view.

FIG. 4 shows a partial view of an orthosis, in section, with the upper part 10, the lower part 20, and the fastening devices 15, 25 formed respectively thereon for releasably fastening the rails. The rails are placed in the fastening devices 15, 25 are fastened to the latter by screws. The screws are screwed into threaded bores inside the upper part 10 and the lower part 20.

The upper part 10 and the lower part 20 are connected to each other about a pivot axis 40 to form a joint device 30. Laterally with respect to the rails (not shown) and to the upper part 10 and lower part 20, two support elements 110, 120 are fastened, in the illustrative embodiment shown screwed, onto the upper part 10 and lower part 20. An upper support element 110 is fastened by screws to the upper part 10 via the bearing plate 200 so as to transmit force and moment. The bearing plate 200 is configured as a separate component and is fastened releasably to the upper part. Correspondingly, a lower support element 120 is fastened to the lower part 20 to transmit force and moment, here with a spacer 90 placed in between. Both support elements 110, 120 are connected pivotably to each other and form a second joint device 130, of which the pivot axis runs coaxially with respect to the pivot axis of the first joint device 30. It is thereby possible that pivoting of the orthosis components about the pivot axis 40 can take place without twisting or jamming.

The two joint devices 30, 130 are arranged laterally and spaced apart from each other, such that a clearance 80 is formed between the joint devices 30, 130. By the doubling of the joint in the region of the rail holders, it is possible, in highly loaded regions of an orthosis, for forces and moments to be effectively taken up and forwarded. The forces occurring in the orthosis are advantageously taken up and forwarded by the frame-like structure in the orthosis joint with the two joint devices 30, 130. The force lines in the longitudinal extent, i.e. from proximal to distal in the extended position, are divided up and run through both the upper part 10 and the lower part 20 with, between these two structural parts, the first joint device 30 and the upper support element 110, the lower support element 120 and, formed therebetween, the second joint device 130. In the region where the support elements 110, 120 are coupled to the upper part 10 and lower part 20, the forces and moments are divided up and brought together, resulting in a closed force train. The laterally arranged joint device 30 thus works as a medial support joint. The distance between the two joint devices 30, 130 corresponds to twice the thickness of the rails. The greater the distance of the joint devices 30, 130 from each other, the more stable the overall orthosis joint, although a very large distance brings disadvantages as regards wearing comfort.

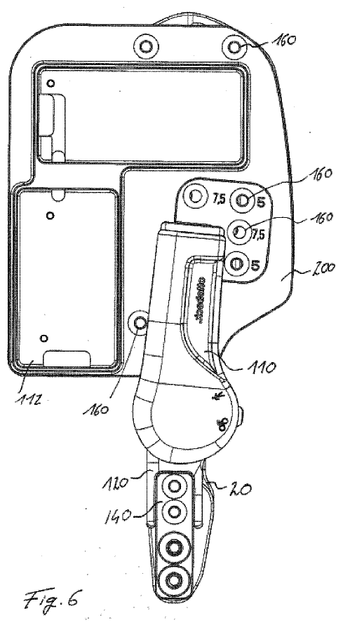

FIG. 5 shows the detail of the orthosis according to FIG. 4 in a plan view from the front. Both the upper part 10 and the lower part 20 and also the upper and lower support elements 110, 120 can be seen with the joint devices 30, 130 laterally spaced apart from each other, i.e. spaced apart from each other along the pivot axis 40. The clearance 80 can be seen between the joint devices 30, 130. To be able to form the clearance 80 and the distance between the joint devices 30, 130 despite a flat configuration of the lower support element 120, a spacer 90 is arranged between the lower part 20 and the lower support element 120, which spacer 90 corresponds to the clearance 80 between the two joint devices 30, 130. Instead of a separate spacer element 90, a clearance 80 can be provided by a corresponding shoulder integrally formed on the lower support element 120. On the upper support element 110, the distance is compensated by the spacer element 95, shown in FIG. 4, in relation to the bearing plate 200.

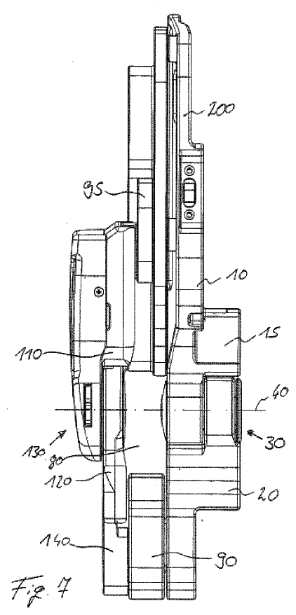

FIG. 6 shows the embodiment according to FIGS. 4 and 5 in an external view, i.e. in a lateral plan view. Beside the upper support element 110, behind a lining and the lower support element 120, the lower part 20 can be seen only partially in the background. Receptacles 112 for further components, for example control devices, sensors or actuators, are formed on the upper support element 110. On the lower support element 120, a kind of rail box is formed, similar to the one in the lower part 20. A fastening strut 140 is inserted in the rail box and connected by screws to the lower support element 120. The strut 140 is connected to the lower part 20 by further screws via the spacer 90 so as to transmit force and moment. By the connection of the lower support element 120 to the lower part 20 via a strut 140 and a spacer element 90, it is possible to achieve easy adaptability to different conditions of use or to different orthoses with an upper part 10 and a lower part 20. The bearing plate 200 is arranged on the upper part 10 (not shown), and the upper support element 110 is in turn fastened to the bearing plate 200. A multiplicity of fastening devices 160 are arranged or formed on the bearing plate 200, which fastening devices 160 make it possible to fasten the upper support element 110 in different orientations and at different positions on the bearing plate 200. Besides a positioning of the upper support element 110 at different locations, it is possible to fasten different upper support elements 110 to the orthosis in order to form a second, parallel joint device 130 and to easily try out or even permanently establish variations in design. In addition, the bearing plate 200 and the multiplicity of fastening devices 160 afford the possibility of add-on components, such as energy stores, control devices, operating elements and the like, being arranged on the orthosis and being coupled to the upper part 10. If the bearing plate 200 is arranged on the lower part 20, a corresponding arrangement of add-on components can be provided on the lower part 20.

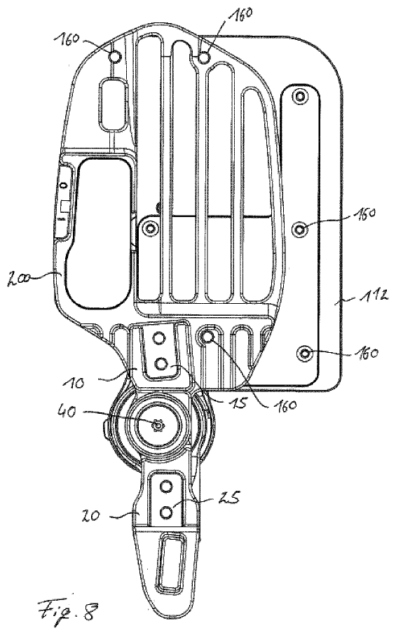

FIG. 7 shows a rear view of the orthosis component. The spacers 90, 95 can also be seen, likewise the upper and lower parts 10, 20, the joint devices 30, 130 and the upper and lower support elements 110, 120. The fastening rail 140 and the receptacle 15 for a thigh rail are also shown, likewise the coaxial pivot axis 40 for the two joint devices 30, 130.

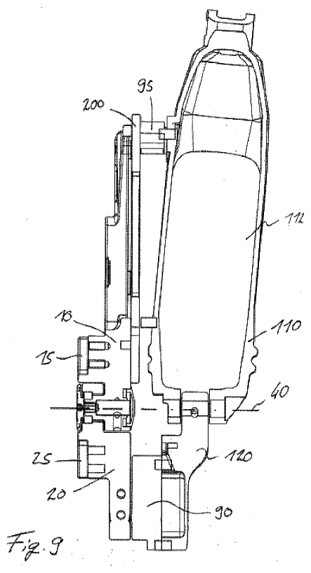

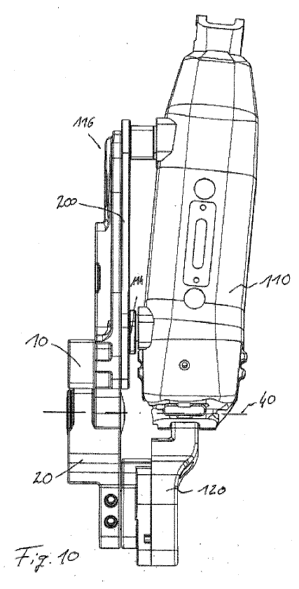

FIG. 8 shows the embodiments according to FIGS. 4 to 7 in a medial view, in which the fastening devices 15, 25 are shown in the form of rail boxes for the rails 60 (not shown), and also the pivot axis 40 running perpendicularly with respect to the plane of the drawing, while the receiving device 112 of the upper support element 110 can be seen in the background. The medial view shows the bearing plate 200 with the multiplicity of fastening devices 160, which bearing plate 200 is fastened to the upper part 10. On the bearing plate 200, which is provided with passages, for example for being able to arrange displaceable fastening elements or for being able to secure further components on the bearing plate, a multiplicity of fastening devices 160 are formed via which different second joint devices 130 can be secured at different locations.

A variant of the invention is shown in FIGS. 9 to 13. The basic set-up corresponds to that of FIGS. 4 to 8, and therefore only the differences will be discussed here. In contrast to the embodiment according to FIGS. 4 to 8, the upper support element 110 is not oriented substantially parallel to the upper part 10 but instead has a slight lateral inclination, which is compensated by the proximal spacer element 95 between the receptacle 112 and the bearing plate 200. The inclination in the medial-lateral direction can be adjusted, for which purpose the joint device 130 can be designed to be adjustable, for example via a pivot joint that permits a medial-lateral pivoting movement, without modifying the orientation of the pivot axis 40 of the second joint device 130. For this purpose, for example, a joint would be provided proximally from the pivot axis 40, namely a joint which in the extended position of the orthosis has a pivot axis that points in the usual walking direction, i.e. runs from anterior to posterior perpendicularly with respect to the plane of the drawing. With such angle adjustability, in which the support is effected via the spacer element 95 or is also adjustable via a thread, an adaptation can be made to the components arranged respectively in the receiving device 112, for example drives, dampers or control devices. In the sectional view according to FIG. 9, the receptacle 112 is configured as a cavity in which, for example, a hydraulic damper or an electromotive drive can be inserted and can be secured therein.

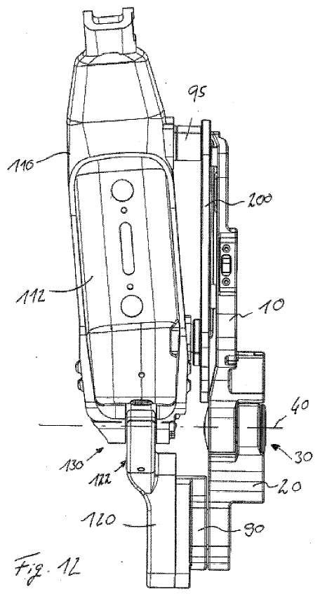

FIG. 10 shows the upper support element configured as a housing in which the receptacle 112 is formed. The upper support element 110 is exchangeably coupled to the upper part 10 at two fastening locations 114, 116 via the bearing plate 200 and via screws or bolts, so as to transmit force.

FIG. 11 is a lateral view showing the receptacle 112 on the upper support element 110 and the receptacle 122 on the lower support element 120. The lower support element 120 does not have a rail box as in the illustrative embodiment in FIGS. 4 to 8, but instead is screwed directly onto the lower part 20 via the spacer element 90. The receptacle 122 on the lower support element 120 is an annular bearing receptacle in which, for example, a bolt of a piston rod for a drive or a damper can be secured, in order to effect or dampen a pivoting of the lower part 20 to the upper part 10 about the pivot axis 40.

The rear view according to FIG. 12 shows the hollow configuration of the upper support element 110 with the receptacle 112, likewise the annular shape of the lower receptacle 122.

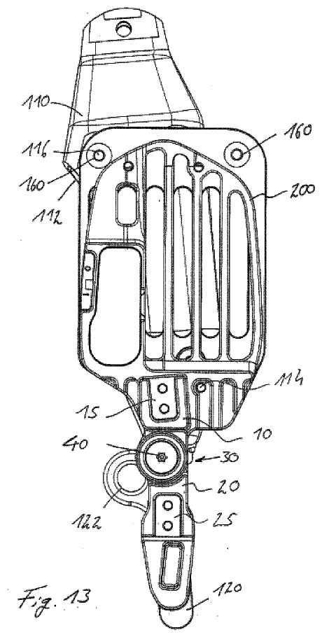

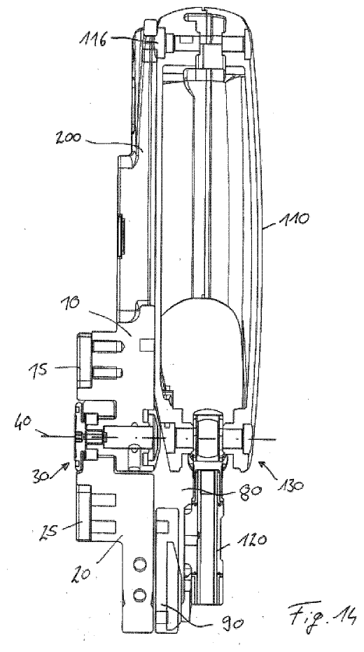

FIG. 13 shows a medial view of the orthosis knee joint with upper part 10 and rail box 15, lower part 20 with rail box 25, and the first joint device 30 for pivoting about the pivot axis 40. The fastening locations 114, 116 and fastening devices 160 for securing the upper support element 110 can be seen on the bearing plate 200.

A third variant of the invention is shown in FIGS. 14 to 18. The set-up of the orthosis joint, which can be configured as a hip joint, knee joint or ankle joint, but also as an elbow joint, basically corresponds to the variant in FIGS. 9 to 13, but with an upper support element 110 oriented substantially parallel to the upper part 10 and the bearing plate 200. On account of the housing-like configuration of the upper support element 110, an arrangement of an upper spacer element is not needed. Furthermore, a clearance 80 between the two joint devices 30, 130 is formed, such that the force lines can run through the first joint device 30 and the second joint device 130 spaced apart from each other in the longitudinal extent of the orthosis. The upper fastening point 116 is relatively far from the pivot axis 40 in order to achieve a high degree of stability of the overall construction. A spacer element 90 is in turn arranged between the lower support element 120 and the lower part 20.

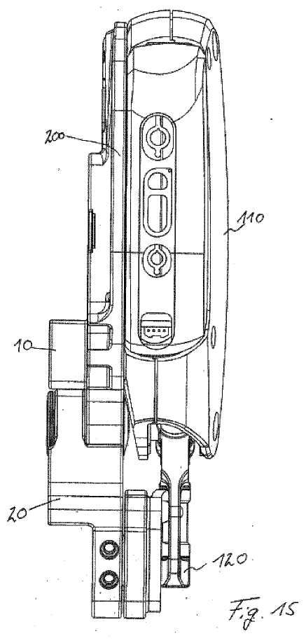

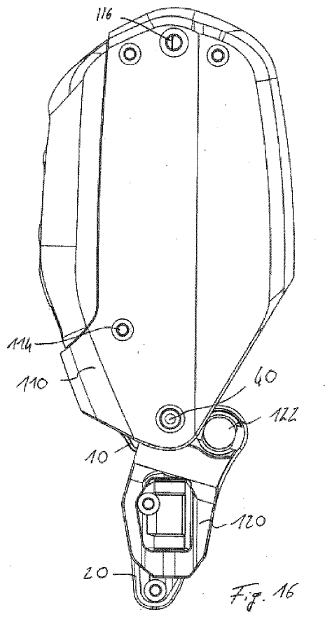

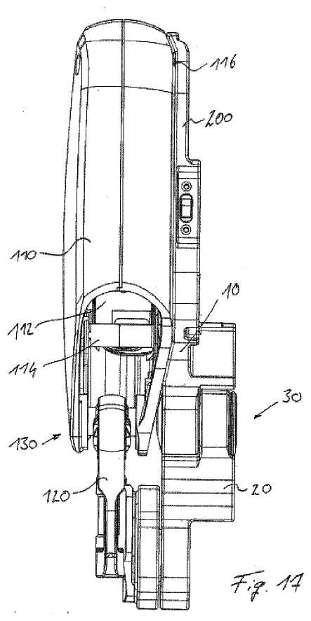

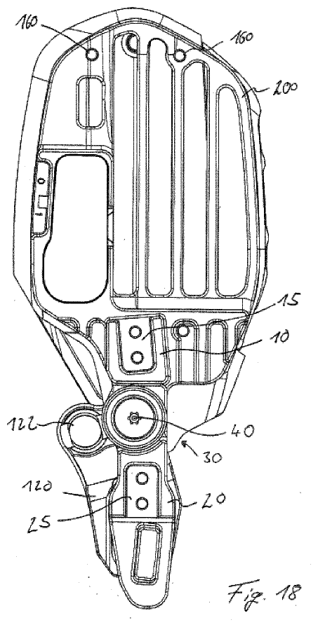

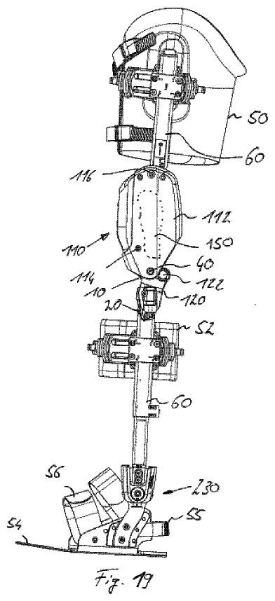

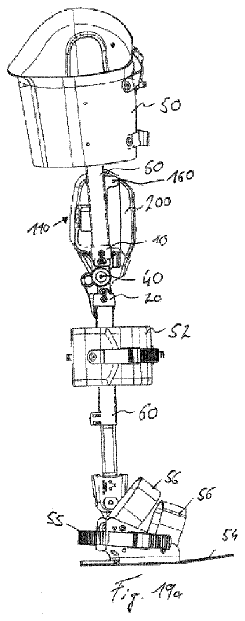

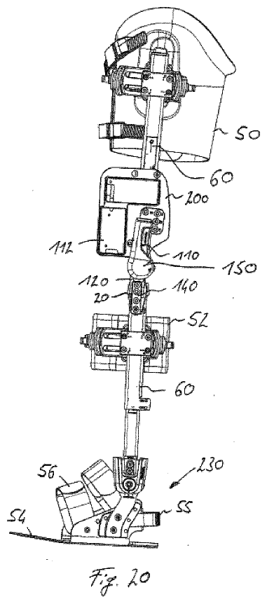

The frontal view according to FIG. 15 shows the compact set-up of the orthosis joint and the shell-like configuration of the upper support element 110. The lateral view according to FIG. 16 shows the upper and lower fastening locations 116, 114 and the receiving device 122 in the form of a bearing ring on the lower support element 120. The rear view according to FIG. 17 illustrates the hollow, housing-like configuration of the upper support element 110, wherein the upper support element 110 is so dimensionally stable that sufficient force transmission and force uptake can take place. The internal view according to FIG. 18 shows the upper part 10 with, adjoining the latter proximally, a plate-shaped enlargement, in order to permit a sufficient contact surface for the fastening of the upper support element 110. The lower part 20 with the rail box 25 is connected with form-fit engagement, and reversibly, by screws to the lower support element 120 and to the receptacle 122 for a bearing pin.

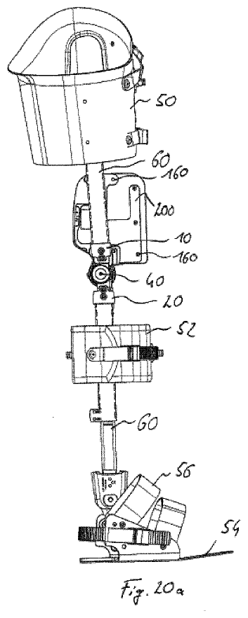

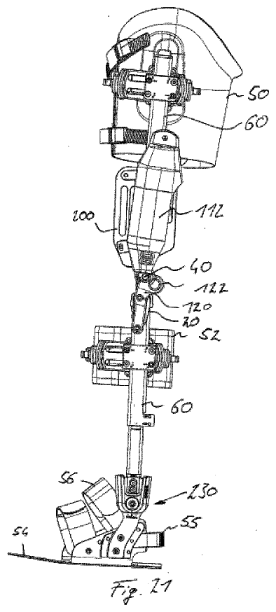

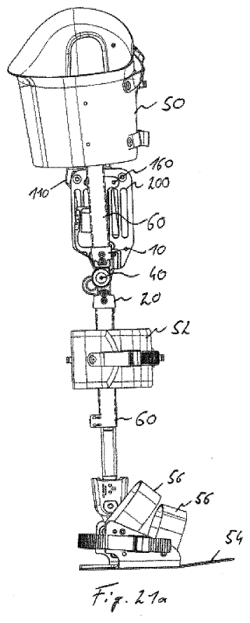

FIGS. 19 to 21 each show side views of an orthosis system with an identical basic set-up, which corresponds substantially to the set-up of an orthosis according to FIGS. 1, 2 and 3. FIG. 19 shows a variant of the embodiment according to FIG. 3, FIG. 20 shows a variant of the embodiment according to FIG. 6, and FIG. 21 shows a variant of the embodiment according to FIG. 11. Common to all the orthoses is the basic set-up with receiving devices 50, 52 and 54, as has been described with reference to FIG. 3, wherein straps 56 have been added in each case in order to fasten and secure the foot on the foot plate 54. In addition, a heel bracket 55 is arranged on the foot part, such that a foot placed on the foot plate 54 is pressed onto or held on the foot plate 54 and also against the heel bracket 55 via the two fastening straps 56. The ankle joint 230, on which functional elements such as adjustable stops or springs can be arranged, is adjoined in the proximal direction by the lower-leg rail 60, on which a lower-leg shell 62 is secured so as to be adjustable both in height and also in the anterior-posterior direction and in an angle position relative to the longitudinal extent of the lower-leg rail 60. The upper part 10 is designed to be pivotable relative to the lower part 20 about the knee joint axis 40. The thigh rail 60, on which the thigh shell 50 is fastened, extends proximally with respect to the upper part 10. The fastening of the thigh shell 50 to the thigh rail 60 is effected, in a manner corresponding to the fastening of the lower-leg shell 52 to the lower-leg rail 60, in an adapter which is displaceable along the longitudinal extent of the thigh rail 60 and via which an adjustment is permitted both of the angle position and also in the anterior-posterior direction and in the medial-lateral direction, i.e. from or toward the thigh rail.

This basic set-up is identical in the system shown in FIGS. 19 to 21. The orthoses differ in terms of different functional elements 150, of which only one is visible. In the embodiment according to FIG. 19, a hydraulic damper device is arranged as functional element 150 in the receptacle 112, said damper device being coupled to the upper part 10 via the receptacle 112 and, for example, the receptacle 122 being connected to the lower part 20 via the lower support element 120.

In the embodiment according to FIG. 20, the set-up according to FIG. 19 is provided not with a hydraulic damper but instead with a driven locking and enabling device as a functional element 150, which is fastened to the upper support element 110. As an alternative to this, a drive can be arranged as functional element in the receptacle 112 according to FIG. 19. The drive permits the initiation of a pivoting movement, or at least supports a pivoting movement, about the pivot axis 40, which is located behind the drive in the illustration in FIG. 20.

FIG. 21 shows the arrangement of a computer-controlled hydraulic damper, as already shown in FIG. 11, on the orthosis. The hydraulic damper as functional element is arranged in the receptacle 112 and can be coupled via a coupling element to the receptacle 122 on the lower support element 120.

In the embodiments according to FIGS. 19 to 21, there is no change to the basic set-up with the receiving devices 50, 52, 54 and 56, the rails 60 and the first joint device 10, 20, the second joint device 130, the functional elements arranged thereon via the respective upper and lower support elements 110, 120, such as hydraulic damper, locking and enabling device, motorized drive or computer-controlled hydraulic damping system are designed such that they have fastening devices which allow the different second joint devices 130 designed as modules to be fastened to the basic set-up and the first joint device 30 to the upper part 10 and lower part 20. For example, in the case of a worsening state of health, a simple hydraulic damper system as shown in FIG. 19 can thus be adapted via a computer-controlled hydraulic damper system according to FIG. 21 to a motor-assisted, active orthosis system or an orthosis with an enabling and locking device according to FIG. 20, or, if the state of health improves, it is possible to change from a driven system via an adaptively controlled orthosis system according to FIG. 21 to a passive orthosis system according to FIG. 19 and then to a locking and enabling system according to FIG. 20.

FIGS. 19a to 21a each show medial views of the embodiments according to FIGS. 19 to 21. It will be seen in the medial view that a fastening device 160 is arranged or formed on the thigh rail 60, to which fastening device 160 the upper support element 110 can be secured via screws, bolts or the like. A corresponding fastening will take place via the lower support element 120 either directly on the lower part 20 or on the lower-leg rail 60. All of the functional elements 150 or all of the support elements 110, 120 have corresponding fastening locations 114, 116, which are configured as bores or pins, for example, and are aligned with the fastening devices 160 or can be brought into engagement with the latter, in order to achieve the second joint device 130 on the first joint device 30 by a coupling between the support elements and the upper part/lower part. The fastening device 160 can also be arranged directly on the upper part 10 or the lower part 20.

1. An orthosis comprising:
an upper part;
a lower part;
a first joint device coupling the upper and lower parts to each other in such a way as to be pivotable about a pivot axis;
devices for receiving a body part or a limb or for securing the orthosis to a body part or a limb are arranged on at least one of the upper part and the lower part;
an upper support element secured to the upper part on one side;
a lower support element secured to the lower part on one side;
a second joint device connecting the upper and lower support elements to each other in such a way as to be pivotable about the pivot axis;
a bearing plate having at least one fastening device for securing the second joint device, the bearing plate being fastened to or formed on at least one of the upper part and the lower part.

2. The orthosis as claimed in claim 1, wherein the first joint device and the second joint device are arranged laterally and spaced apart from each other.

3. The orthosis as claimed in claim 1, wherein the support elements are connected to each other releasably via the second joint device.

4. The orthosis as claimed in claim 1, further comprising fastening devices for the releasable fastening of rails or devices for receiving a body part or a limb, the fastening devices being formed or arranged on the upper part and the lower part.

5. The orthosis as claimed in claim 1, further comprising receptacles for at least one of a damper, an enabling and locking device, a drive, a control device, a spring store, an extension assist and a sensor are arranged or formed on the upper support element and the lower support element.

6. The orthosis as claimed in claim 1, wherein the support elements are fastened releasably to the upper part and the lower part via the bearing plate.

7. The orthosis as claimed in claim 1, wherein at least one of the upper support element and the lower support element is designed to be adjustable in terms of angle to the pivot axis.

8. The orthosis as claimed in claim 1, wherein the orthosis is designed as a cross-knee orthosis, knee-ankle-foot orthosis (KAFO), or ankle-foot orthosis.

9. The orthosis as claimed in claim 1, wherein the support elements are secured to the upper part and the lower part to transmit force and moment.

10. The orthosis as claimed in claim 1, wherein the support elements are made from metal or a fiber-reinforced plastic.

11. The orthosis as claimed in claim 1, wherein at least one of the support elements is designed as a functional element, and such a functional element is fastened to at least one support element.

12. A system composed of an orthosis as claimed in claim 1 and of a plurality of different functional elements, which can be secured to the bearing plate at the same fastening devices.

13. The system as claimed in claim 12, wherein the functional elements or the support elements have fastening locations which are arranged corresponding to a fastening device for the fastening locations for fastening to the upper part and the lower part.

14. The system as claimed in claim 12, further comprising at least one spacer element is arranged between the respective functional element and at least one of the upper part and the lower part or the bearing plate.

15. The system as claimed in claim 12, wherein the functional element is secured exchangeably to the bearing plate.

16. The system as claimed in claim 12, wherein the upper support element and the lower support element are assembled to form a module.

17. A method for adapting an orthosis as claimed in claim 1 to a patient, on whom the orthosis is fastened to a body part or limb via the devices for receiving or for securing a body part or a limb, said method having the steps of:
selecting a second joint device from a multiplicity of different second joint devices, and
connecting an upper and lower support element, which are connected to each other via the selected second joint device, to the upper part and the lower part of the first joint device.

18. The method as claimed in claim 17, wherein a first joint device is selected from a plurality of different first joint devices and is connected to the devices for receiving or securing a body part or limb.

19. An orthosis comprising:
an upper part;
a lower part;
a first joint device pivotally coupling the upper part to the lower part about a first pivot axis;
receiving devices configured to receive a body part or a limb or for securing the orthosis to a body part or a limb, the receiving devices being arranged on at least one of the upper part and the lower part;
an upper support element secured to the upper part;
a lower support element secured to the lower part;
a second joint device pivotally connecting the upper support element to the lower support element about a second pivot axis;
a bearing plate having at least one fastening device and being fastened to or formed on at least one of the upper part and the lower part.

20. The orthosis as claimed in claim 19, wherein the first joint device and the second joint device are arranged laterally and spaced apart from each other.
